(19) United States
(12) Patent Application Publication
(10) Pub. No.: US 2023/0158199 A1
(43) Pub. Date: May 25, 2023

(54) INTEGRATED SYSTEM FOR SANITIZATION AND EMERGENCY LIGHTING OF ROOMS

(71) Applicant: BEGHELLI S.P.A., VALSAMOGGIA (BO) (IT)

(72) Inventor: Gian Pietro BEGHELLI, VALSAMOGGIA (BO) (IT)

(21) Appl. No.: 17/906,674

(22) PCT Filed: Apr. 15, 2021
(86) PCT No.: PCT/IT2021/050113

§ 371 Date: Sep. 19, 2022

(30) Foreign Application Priority Data
Apr. 17, 2020 (IT) 102020000008248
May 29, 2020 (IT) 102020000012892

(51) Int. Cl. A61L 9/20; A61L 9/12; F21S 9/02

(52) U.S. Cl. CPC A61L 9/205; A61L 9/122; F21S 9/024; A61L 2209/12; A61L 2209/111; A61L 2209/212

(57) ABSTRACT

An integrated system for sanitization and emergency lighting of rooms, comprising: - an external housing (23), which includes a white light source (13) with an optical system, apt to realize an emergency lighting, a battery (18) for operating said emergency lighting in the absence of a main power supply, - a chamber (19), provided with reflecting walls and internal lens shade baffles (16), in which a photo-catalytic reactor (20) and/or a UVC light source are housed, and - a fan (22), of axial or tangential type, which creates inside said chamber (19) a circulation or flow of forced air (F) taken from outside, upstream of said chamber (19) through inlet slots (17), and reintroduced outside, downstream of said chamber (19) through outlet slots (10), and - an electronic control circuit (14), which controls the operation of the white light source (13), the UVC light source and/or the photo-catalytic ballast (20), the battery (18) and the fan (22), characterized in that, inside said chamber (19), an ambient light sensor (11) is housed, aimed at measuring the brightness of the environment in which said system is positioned.

The present invention generically relates to an integrated system for sanitization and emergency lighting of rooms.

More particularly, the invention relates to an emergency lighting fixture integrating an ultraviolet sanitizing device, wherein air, which is circulated by a fan, passes through a chamber within the lighting fixture irradiated by ultraviolet C-band (UVC) energy, together with, or alternatively, a photo-catalytic reactor activated by ultraviolet UVA-UVB light.

The literature regarding sanitization using UV light is long-standing, and four solutions are typically used for room sanitization:
1. UVC lamps that illuminate the upper portions of the rooms taking the utmost care not to radiate energy towards the occupants for safety reasons;
2. systems of sanitization of surfaces by direct irradiation in the absence of people in the area;
3. air treatment systems with forced ventilation to be applied inside the premises;
4. air treatment systems integrated into building air conditioning (HVAC) equipment, in which UVC lamps are placed inside the ventilation ducts. Mercury discharge tubes or lamps have a peak emission at 254 nm, very close to the wavelengths at which they are most effective in deactivating microorganisms such as viruses and bacteria. At the state of the art mercury discharge tubes or lamps have a high energy efficiency and about ¼ of the electrical power supply is converted into UVC radiation.

UVC LEDs, on the contrary, have today a low efficiency, an order of magnitude lower, however as soon as technological evolution will make available more efficient solid state sources (LEDs) it will be trivial to replace the discharge tube with the LED matrix.

UVC LEDs have the advantage of being able to tune the wavelength of the emitted light and possibly to build a matrix composed of several components with different wavelengths; in this way, the sanitizing device could be of type “multiband” and hit more effectively microorganisms different from each other, each LED being tuned to hit more effectively specific links of DNA and/or RNA chains.

On the other hand, the operating principle of a photo-catalyst is as follows: a semiconductor material, such as titanium dioxide (TiO2), is irradiated with light suitably tuned to the band-gap characteristic of the semiconductor itself. Electrons excited by the incident photons move into the conduction band, leaving gaps in the valence band of the semiconductor.

On the surface of the catalyst, electrons can thus combine with electron “acceptor” elements in a reduction reaction, while gaps can combine with electron “donor” elements in oxidation reactions.

Overall, oxidation-reduction reactions result in three mechanisms for deactivation of chemical species and pathogens:
1) Direct reduction process, whereby the pollutant species (Pollutant) combines directly with either an electron or a gap and results in an inorganic product.

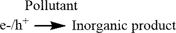

2) Two-stage process, according to which the presence of free electrons and gaps in the presence of oxygen and water generates OH and O2 radicals, which, combining with pollutants, inactivate them producing stable inorganic compounds.

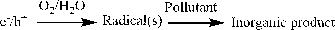

3) Three-stage process, according to which radicals can be transformed into ROS (Reactive Oxygen Species), which then combine with pollutants to produce stable inorganic compounds.

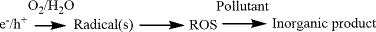

The effectiveness of photo-catalysts depends on their chemical and physical characteristics, in particular on the morphology of the surface on which the chemical reactions take place; for example, the effective active surface, with the same size (porosity), and the size of the grains that make up the active surface are important.

Photo-catalysis has been shown to disinfect a variety of pathogens, including bacteria, fungi, and viruses. The mechanism of deactivation is based on the interaction of ROS species with the cell membrane of microorganisms, which is compromised and the microorganisms killed.

In the photocatalysis system implemented by this integrated system, air, circulated by a fan, passes through a photo-catalytic cell inside the device; the oxidation and reduction reactions that take place inside the chamber purify the air by transforming some of the chemicals present and sanitize the air by deactivating viruses and bacteria, as well as remove unpleasant odors from the air itself.

The device can also optionally contain an ozone generator placed downstream of the photo-catalytic cell, which can be turned on by a timer or by a radio remote control to be activated only when people are absent; for this purpose, the device also integrates a people presence sensor (for example, a microwave radar or a PIR sensor) to turn off the ozone generator when people are detected present.

With respect to emergency lighting of rooms and sanitization of rooms, it is currently contemplated to install two devices, such as an emergency lamp and an air purification and sanitization device; in the case of using ozone, it is further necessary to provide for the installation of three devices, namely an emergency lamp, an air purifier and an ozone generator.

The purpose of the present invention is to provide an integrated system for sanitizing and for emergency lighting of rooms, which allows in a single mechanical structure to provide adequate safety lighting of rooms in the event of a blackout and which, at the same time, contributes to the purification and sanitization of the air of a room conveyed into the system, by means of the germicidal action of an integrated UVC source and/or a photo-catalytic reactor. Another purpose of the present invention is to produce an integrated system for the sanitization and emergency lighting of rooms, which allows to sanitize the air in the rooms in which it is installed, deactivating a certain percentage of pathogenic organisms at each cyclic passage of the air itself inside the system. A further scope of the invention is to realize an integrated system for the sanitization and emergency lighting of rooms, which allows reducing the diffusion of bacteria, fungi and viruses in the air and at the same time to improve the air quality by reducing the quantity of volatile organic compounds. A further purpose of the invention is to realize an integrated system for sanitizing and emergency lighting of rooms, which also allows carrying out a complete sanitization of the environment in the absence of people.

These and other purposes are achieved by an integrated system for sanitizing and for emergency lighting of environments, according to the appended claim 1. Other detailed features can be found in the dependent claims.

Advantageously, the present invention consists of integrating an air sanitizing system using UVC radiation sources and/or photo-catalytic reactor within an emergency lighting fixture, with which advantageously they share housing and internal electrical control and operating parts.

The new system, being bound to the lighting positioning, takes advantage of this constraint by placing the purifier and/or sanitizing device in the most suitable positions for the treatment of the air inside the premises; in fact, the emergency lamps are always installed high up, in optimal positions for the diffusion of light and consequently also for the treatment of the air.

The new system therefore includes an emergency luminaire containing an internal chamber illuminated by a UVC source and/or a photo-catalytic reactor (consisting of a micro-catalyst and a UVA-UVB source), through which air circulation is forced by a ventilation system.

Conveyed air carries aerosols that include pathogenic microorganisms (bacteria, viruses, and fungi) and volatile chemical compounds VOCs (Volatile Organic Compounds).

Photo-catalysis and/or UVC radiation sources, at wavelengths ranging from approximately 250 nm to approximately 280 nm (having the greatest germicidal capacity), are capable of neutralizing many chemical species and pathogenic organisms.

The system is sized so as to treat every day one or more times the entire volume of air in the environment in which it is installed by supplying the air in the environment with one or more doses of germicidal radiation; it can optionally contain an ozone generator, which can be activated by the user, and which is protected by automatic mechanisms to prevent it from being turned on in the presence of people in the environment in which the system is installed.

In particular, the ozone generator, which can be integrated in the emergency lighting fixture, can be of two types
with UVC lamp; in this case, ozone is generated by the radiation emitted at wavelengths of about 185 nm that low-pressure mercury discharge lamps naturally emit if the quartz of the discharge tube is not filtered at these wavelengths;
with corona discharge generation; in this case, the corona generator consists of a dielectric plate placed between two electrodes to which high voltage is applied so as to cause a corona discharge and ozone is formed by the electrical discharge spread over the entire area of the dielectric, which allows the conversion of oxygen in transit into the molecular form O3.

Finally, the system is very compact, as integration into the emergency lamp allows multiple safety functions to be combined within the same device, reducing the proliferation of different objects and devices within buildings.

Additionally, the integrated system has a lower cost than the sum of the separate devices (emergency lamp, photocatalysis and/or radiation purifier, ozone sanitizing device), since it uses some common parts.

Further purposes and advantages of the present invention will be more clearly understood from the following description of preferred, non-limiting embodiments of the integrated room sanitizing and emergency lighting system according to the present invention, and from the appended drawings, in which:

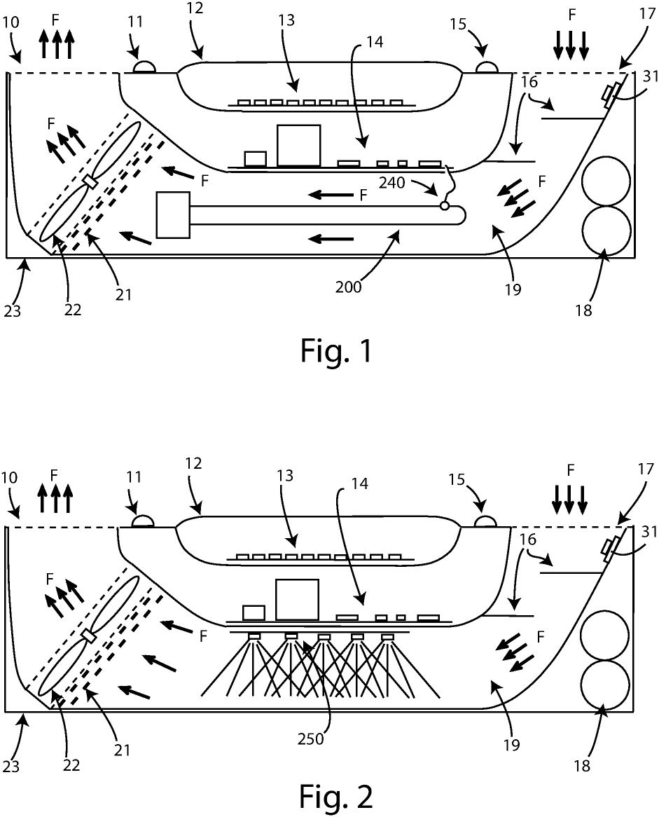

- FIG. 1 shows a longitudinal section of the integrated system for sanitization and emergency lighting of rooms, having a UVC source comprising one or more tubes or low pressure discharge lamps, according to a first embodiment of the present invention;

- FIG. 2 shows a longitudinal section of the integrated system for sanitizing and for emergency lighting of rooms, according to a second embodiment of the present invention, equipped with a UVC source comprising a strip containing a matrix of UVC LEDs;

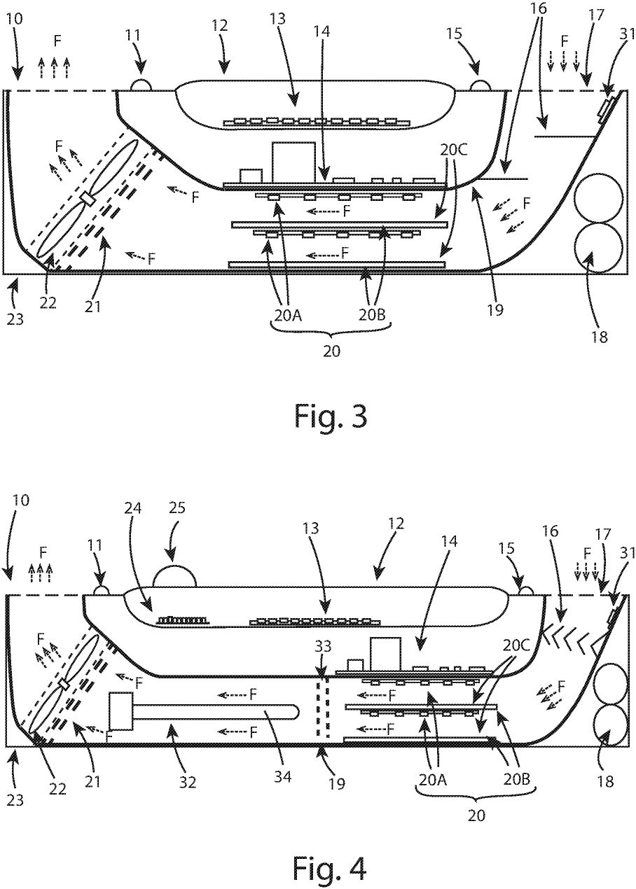

- FIG. 3 shows a longitudinal section of the integrated system for sanitizing and for emergency lighting of rooms, with a photo-catalytic reactor and without an ozone generator, according to a third embodiment of the present invention;

- FIG. 4 shows a longitudinal section of the integrated system for sanitization and emergency lighting of rooms with photo-catalytic reactor and ozone generator realized with 254 nm main emission lamp unfiltered in wavelengths below 200 nm, according to a fourth form of embodiment of the present invention;

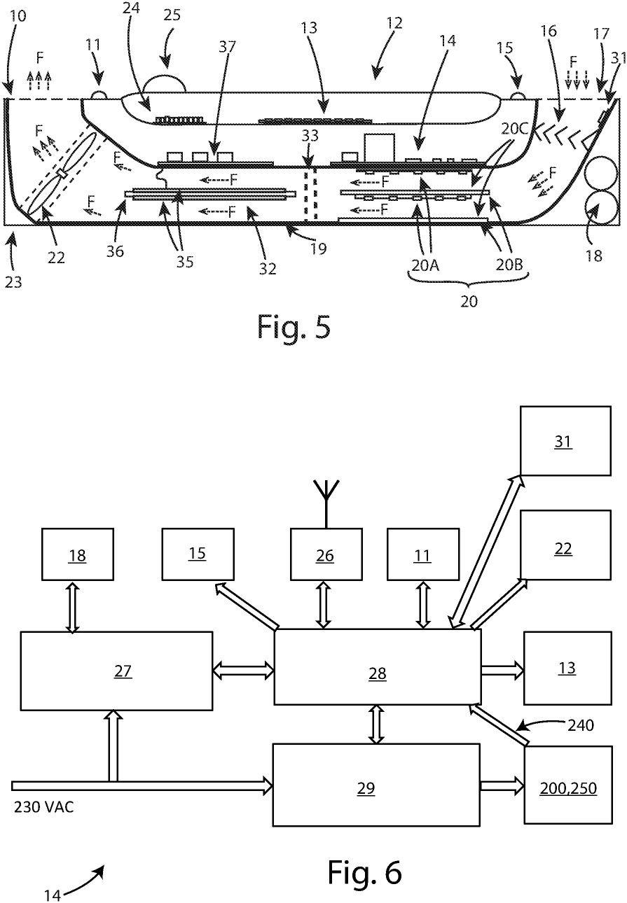

- FIG. 5 shows a longitudinal section of the integrated system for sanitization and emergency lighting of rooms, according to a fifth form of embodiment of the present invention, with photo-catalytic reactor and ozone generator realized with corona discharge plate;

- FIG. 6 shows a block diagram illustrating an example of an electronic control circuit of the integrated system for sanitization and emergency lighting of rooms, of embodiments of FIGS. 1 and 2, according to the invention;

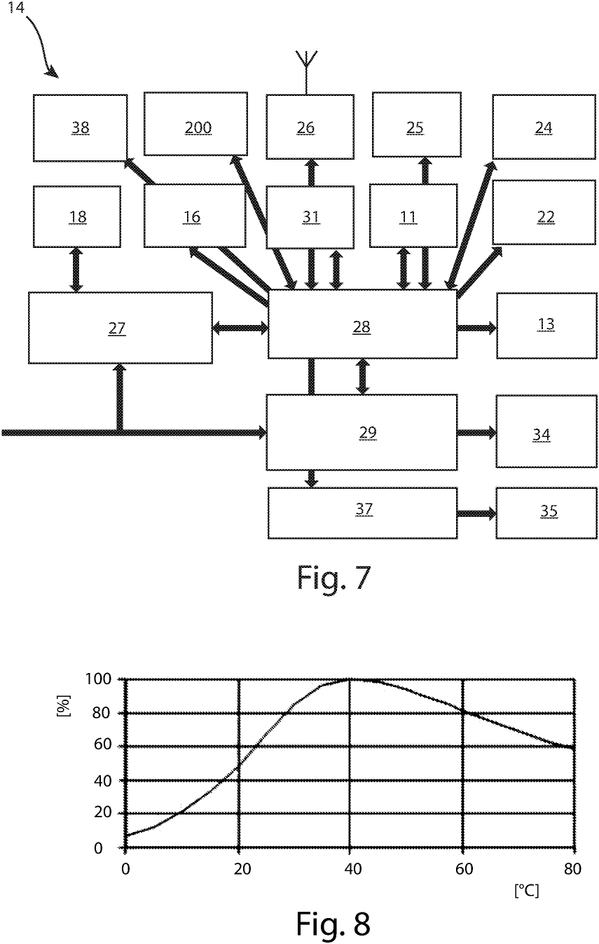

- FIG. 7 shows a block diagram illustrating an example of an electronic control circuit of the integrated system for sanitizing and for emergency lighting of rooms, of the embodiments of FIGS. 3-5, according to the invention;

- FIG. 8 shows a Cartesian diagram of the UVC emission performance curve of low pressure mercury discharge tubes as a function of the operating temperature of the tubes.

In order to facilitate understanding of the figures, the represented embodiments alternatively comprise the photocatalytic reactor or the UVC radiation source, but the description is intended to be extended also to embodiments of the invention comprising both, as moreover already fully specified in the introductory section of the present application.

With reference to the figures mentioned, the integrated system according to the present invention includes an outer housing 23, made, for example, of a technopolymer plastic, within which the following parts are incorporated:
a white light source 13, for example comprising an array of LEDs, with an appropriate optical system 12 for performing emergency lighting;
a battery 18 for operating the emergency lighting in the absence of the main power supply;
a chamber 19, preferably made of metallic material or material resistant to UV radiation and provided with reflective walls suitable for reverberating UV radiation inside and internal shade baffles 16 to prevent the escape of UV radiation, in which are housed:
   a UVC light source, which may be a low-pressure mercury discharge lamp 200 (FIG. 1) or a UVC LED array 250 (FIG. 2), and/or
   a photo-catalytic reactor 20, such as a TiO2 type, activated by UVA-UVB radiation and equipped with UVA-UVB plates 20A, flat photo-catalytic plates 20B, and photo-catalytic surfaces 20C (FIGS. 3-5);
a fan 22, of axial or tangential type, which creates inside the chamber 19 the forced circulation of air (flow F) taken from outside through the inlet vents 17 and reintroduced outside, downstream of the chamber 19, through the outlet vents 10;
a light sensor 11 to measure the brightness of the environment in which the device is positioned;
an air quality sensor 31, positioned inside the chamber 19, near the air inlet vents 17, capable of functioning and carrying out measurements correctly even with the fan 22 switched off. This sensor 31 measures the average concentration of volatile organic compounds VOC or VOX (acetone, methanol, ethanol, hydrocarbons, etc.);
an electronic circuit 14 governing the operation of the various devices, such as the white light source 13, the UV light source 200 and/or the photocatalytic reactor 20, the battery 18, the fan 22, optical and acoustic signals, sensors, and the optional ozone generator 32;
optionally, an ozone generator 32 placed downstream of the chamber 19, and realized by means of a UVC discharge lamp 34, in particular low-pressure mercury, unfiltered, at 185 nm, or by means of high-voltage electrodes 35, placed on either side of a dielectric 36 (e.g., a ceramic substrate) and employed for a corona discharge, fed by a corresponding high-voltage circuit 37, wherein the metal inner walls of the chamber 19 constitute the other pole of the generator;
suitable sensors 24 (microwave), 25 (PIR) for the presence of people (optional), either PIR or microwave radar;
a transceiver radio module 26 of BLE or IEEE802.15.4 type for sending information to remote actuators and for remote control of system operation;
optical (such as signaling LED 15) and acoustic (buzzer 38) signaling devices.

A direct air flow F from right to left is shown in the figures, but the direction can be reversed from left to right depending on the specific sizing of the duct and sanitizing chamber 19.

In any case, the air flow F is preferably oriented so as to avoid sending the ozone created by the optional generator 32 into the area of the chamber 19 occupied by the UV sources or the photo-catalytic cell.

The UVA-UVB light source of the photo-catalytic reactor or photo-catalyst 20 preferably comprises UVA-UVB LED plates 20A emitting in the bands from 300 to 450 nm, depending on the characteristics of the catalyst. The catalyst may be made from titanium dioxide (TiO2) or other materials, such as ZnO, CeO2, ZrO2, SnO2, WO3.

The photo-catalytic reactor 20 described and illustrated in the attached FIGS. 3-5 further comprises a stack of flat photo-catalytic plates 20B, each illuminated by its own UVA-UVB LED plate 20A crossed by the air to be purified and/or sanitized. Different forms of the photo-catalytic reactor 20 may, however, be used alternatively, such as a cylindrical reactor internally illuminated by an LED strip located in the center or on an inner side, with the photo-catalytic material deposited within the cylinder.

The UVC light source may comprise a low-pressure mercury discharge lamp (shown in detail in the attached FIG. 1 and indicated by 200) or a strip or band containing an array of UVC LEDs (shown in detail in the attached FIG. 2 and indicated by 250) with emission wavelengths at 270 or 280 nm or according to a mix of these wavelengths or others nearby.

To maximize the energy dose that the conveyed aerosol absorbs at each crossing of the chamber 19 illuminated by the UVC source, sizing is done according to the following criteria:
largest possible illuminated zone volume;
maximum available power of UVC radiation in the allocated volume.

In fact, the germicidal dose Eg (which dimensionally corresponds to the UVC energy density to which microorganisms are subjected) is expressed in J/sqm and is obtained as the product of the radiant power Pg (W/sqm) in the illuminated zone and the time T of transit through the zone itself, i.e. Eg = Pg x T.

It is evident that the greater the illuminated volume of chamber 19, with the same air flow, the greater the time of transit by the aerosol.

From the scientific literature we find indications of minimum necessary doses between a few J/sqm and 100 J/sqm, depending on the susceptibility of viruses and bacteria that we want to kill.

Using lamps from 5 to 20 W as UVC sources, average power densities of a few tens of W/sqm are typically obtained in the illuminated inner chamber. Furthermore, considering, for example, a transit time on the order of 1 second, the doses are therefore several tens of J/sqm.

For the sizing of the fan 22 in the typical size of an emergency lamp (300 x 150 x 50 mm), it is advantageous to have airflows between 1 and 5 cubic meters per hour, so as to ensure the complete air exchange of a room one to a few times per day.

At the same time a reduced flow offers the advantage of a high level of silence of the device and the maintenance of a high dose of radiation at each transit of the chamber 19; in fact, a low intensity air flow is equivalent to a high transit time of the chamber 19.

The sanitizing chamber 19 is preferably made of metal with reflective inner walls so as to minimize any shaded areas and to ensure that the aerosol is treated by the UV light source 200, 250 and/or the photocatalytic reactor 20 during the entire residence time in the chamber 19 itself, in order to enhance the sanitizing effect.

Suitable shade baffles 16 are positioned in the air inlet and outlet ducts, which prevent the escape of UV radiation from the chamber 19 to the outside, while at the same time ensuring the lowest possible pressure drop in the air duct. Appropriate separating plates 33 may also be introduced between the UV source (UVC light source 200, 250 and/or photo-catalytic reactor 20) and the ozone generator 32 when present, to avoid contamination or electrical discharge.

Any internal plastic parts, such as fan blades 22, are protected from UV radiation by staggered metal grids 21, which limit the intensity of UV light directed at such parts (which could be responsible for premature aging of such materials).

The apparatus may advantageously incorporate a pyroelectric sensor 25 for the presence of people, as an alternative to a microwave radar 24 for the same function. In the event that it is desired to increase the safety of detecting people in the environment, the two sensors 24, 25 may both be present and used in an “OR” mode for inhibition of the ozone generator 32, if present, following detection of people by only one of the two sensors 24, 25.

Advantageously, the system also incorporates an air quality sensor 31, positioned inside the chamber 19 and, in particular, near the air inlet vents 17, capable of functioning and performing measurements correctly even with the fan 22 turned off; the sensor 31 measures the average concentration of volatile organic compounds VOC or VOX, (acetone, methanol, ethanol, hydrocarbons, heptanes, toluene, xylene, etc.).

The sensor 31 may be activated to turn on the photo-catalytic reactor 20 automatically when the concentration of VOCs exceeds a certain value in the environment, which also typically corresponds to the prolonged presence of people in a room that is not well ventilated.

The block diagrams in FIGS. 6 and 7 illustrate the electronic circuit 14 controlling the system, which integrates all the control and regulation functions of the various constituent electrical elements.

Specifically, the microprocessor 28 of the electronic circuit 14 governs the operation of the entire system and coordinates the various subsystems as follows:
turns on and off the white LEDs of the emergency light source 13;
turns on and off the UVA-UVB LEDs 20A of the photo-catalytic reactor 20 and/or the discharge tube 200 used as the UVC source, possibly adjusting the power of the tube itself and the speed of the fan 22 to maintain the temperature at a predetermined level;
measures light in the environment, via the sensor 11, to potentially determine night periods and the on/off strategy of the UVC source 200, 250 (powered via an electronic ballast 29) and/or the photocatalytic reactor 20 and/or the ozone generator 32 (if present);
manages the microwave sensor 24 and the pyroelectric sensor (PIR) 25 to detect the presence of people in the environment and consequently switch off the ozone generator 32 (if present);
analyzes the air quality sensor 31 for the concentration of volatile organic compounds (VOC) and, if present, automatically turns on the photo-catalytic reactor 20 and the fan 22;
manages the switching on and off of the ozone generator 32 with corona discharge (if present), controlling the switching on and off of the discharge tube 34 (fed by an electronic ballast 29) or of the electrodes 35 for ozone generation and post-sanitation;
manages the switching on and off of the fan 22;
manages the acoustic signaling device (buzzer 38) to indicate to the user the danger linked to the presence of ozone;
manages and diagnoses the power supply and/or battery charger block 27 and the battery status 18 depending on the availability of the emergency lighting function;
drives the signaling LEDs 15 indicating to the user a series of statuses, such as power supply present, sanitizing device on, ozone generator 32 in operation, sanitizing device failure, emergency function failure;
manages a radio transceiver 26, such as Bluetooth® BLE, for communication with external devices (e.g. smartphones or tablets) for configuration and command or manages an IEEE802.15.4 radio, such as the “Beghelli® FM”; in equivalent solutions the radio transceiver 26 can be replaced by a wired communication interface that exploits the communication capability of centrally controlled emergency lighting systems (e.g. CT Beghelli®, or Cablecom Beghelli®) to receive commands from a centralized control system. In addition, the radio transceiver 26 can send to a relay actuator the command to turn on and off an outdoor fan for air exchange in the room after the ozone sanitization process has been completed.

The functioning of the integrated system for sanitization and emergency lighting of rooms is basically the following.

In the presence of 230Vac power supply, once installed, the system activates the photo-catalytic reactor, through the lighting of the UVA-UVB LEDs 20A, and/or the UVC source 200 and the fan 22. in order to start the sanitizing and air purification function.

Simultaneously, the battery 18 related to the emergency lighting is charged and maintained in a charged state.

In versions that include the ozone generator 32, the user can activate this function via a BLE radio command given by a smartphone equipped with an appropriate application, or via an IEEE802.15.4 radio command from a remote management system of centralized emergency lighting control, possibly cloud-based (e.g., the Beghelli® nuBe).

Alternatively, the ozone generation function can be controlled by a daily or weekly timer programmed during system configuration.

In any case, the ozone generation function is not activated as long as the presence of people is detected by one of the sensors 24, 25 present in the room.

In order to avoid that in any case the accumulation of ozone at small intervals can be harmful, the generation function is activated only if the presence of people is not detected for at least 60 minutes and, in case of detection of the presence of people, the generation of ozone is immediately stopped and the acoustic signaler (buzzer 38) with fast intermittence (2 Hz) is activated to indicate to leave the room or to ventilate.

When the ozone generator 32 is turned on, the signaling LED 15 flashes with red color at high frequency (2 Hz).

It is possible to configure the product in such a way that a beep is also emitted by the buzzer 38 every 2 seconds (0.5 Hz), in the absence of people present, to signal the emission of ozone.

As soon as a sensor 24, 25 detects the presence, the beeper 38 increases the frequency to 2 Hz to insistently signal the danger.

The two versions of ozone generator 32 described in the present invention differ in that one (FIG. 4), the one with UVC source 34, is featured by the generation of smaller amounts of ozone, but has enhanced germicidal efficacy when added in cascade to the germicidal action of the UVC light from photo-catalytic reactor 20; the second version, with corona discharge ozone generator 32 (FIG. 5), can produce larger amounts of ozone.

Ozone is useful for sanitizing the entire environment, including the surfaces of objects placed in the room that otherwise would not be sanitized by the airflow through the chamber 19.

The ozone generated becomes itself a germicidal agent able to propagate in the environment, acting on the surfaces and on the objects it meets.

The purification and/or sanitization mode can be configured via the BLE radio interface with an APP from a smartphone or tablet, or from a remote management system for centralized emergency lighting control, possibly cloud-based (e.g., Beghelli® nuBe).

The speed of the fan 22 can be defined by choosing, for example, between a slow speed corresponding to lower airflow F (and therefore less frequent air exchange) and quieter operation, or a higher speed with higher airflow, but higher fan 22 noise.

It is also possible to choose the daily sanitizing switch-on cycle, for example between “always on”, “on for 4 hours a day” or “on for 8 hours a day” or the automatic mode managed by the air quality sensor 31.

Finally, it is possible to determine the modes and times when the ozone generator is switched on 32.

The default mode of operation for the UVC source with discharge tube 200 is typically with 8-hour daily turn-on to maximize the life of the tube itself, which is on the order of 15000 hours, so that it has a useful life of at least 4-5 years. The communication interface also allows the emergency mode of operation to be configured in accordance with the customs of emergency luminaires.

In the absence of the main power supply, the sanitizing function is turned off and the battery 18 is used to power the light source (LED) of white light 13 for a defined time.

The sanitizing function is kept on when mains power is present according to the cycle selected by the user.

The low pressure mercury discharge tubes or lamps 200 have a UVC emission efficiency curve that is significantly dependent on the operating temperature of the tubes, as shown in the attached FIG. 4.

In this regard, a temperature sensor 240, located on the discharge tube or lamp 200, is advantageously employed in the system.

The microprocessor 28 measures the temperature and, acting primarily on the driving power of the discharge tube or lamp 200, maintains the temperature at the optimum value for having the maximum UVC power emitted, causing the tube or lamp 200 to work at the optimum point (in the case of the attached FIG. 8, around 40° C.).

If necessary, the microprocessor 28 also acts on the speed of the fan 22 to adjust the temperature to the optimum value; this function allows the maximum germicidal sanitizing efficacy to be maintained under any operating environmental conditions.

As mentioned, the system can also be equipped with a sensor 11 for measuring ambient light, which has the purpose of identifying the day/night cycle and activating, for example, the sanitizing function during the day and turning it off during the night, according to the set cycle.

In this way, maximum silence is guaranteed at night (fan 22 off) and the system is automatically activated only during daylight hours.

Alternatively, the system can be configured with different cycles according to specific user requirements and/or always synchronized to the day/night cycle. As an alternative to the ambient light sensor 11, the microprocessor 28 can be equipped with an astronomical clock, calibrated at the factory with a small battery to operate with precise knowledge of the time of day and time of year and thus activate the most appropriate sanitizing cycle moment by moment.

In a more complete and versatile realization, the device also incorporates the air quality sensor 31 based on the measurement of the concentration of VOC to automatically activate the sanitizing cycle when a certain overall concentration is exceeded.

In this way the sanitizing device is automatically activated in the presence of occupants of the room in which it is installed when the pre-set air pollution levels are exceeded, exerting its germicidal sanitizing action when people are present in the environment.

Regarding the ozone generation function, the system generates a radio command when the ozone generation cycle has been completed; this radio command is sent to a relay actuator and the radio relay turns on a fan external to the system, which performs the air exchange in the room to remove the ozone before the people come back in. The control is timed by the system, and the time the external fan is turned on is configurable via the radio interface itself.

In a further different mode of operation, the ambient light sensor 11 is used as a sensor for the movement of people within the same room, based on the detection of sudden changes in ambient brightness; in this way, the microprocessor 28, by means of the light sensor 11, is able to detect the entry of an occupant into the room and activate a sanitization cycle in correspondence to this event and then put itself in a state of quiet in the absence of movement detected by the light sensor 11 itself.

The information from the air quality sensor 31 and the light sensor 11 can finally be used in synergy with each other to make the sanitization strategy even more refined.

In essence, a “smart” sanitizer with low-cost sensors that are completely non-invasively integrated into the emergency lighting fixture is thus realized.

The invention thus conceived and illustrated is susceptible to modifications and variations, all of which fall within the inventive concept of the appended claims. Furthermore, all details may be replaced by other technically equivalent elements.

Finally, the components used, provided that they are compatible with the specific use, as well as the dimensions, may be any according to the requirements and the state of the art.

Where features and techniques mentioned in the claims are followed by reference marks, such reference marks have been included for the sole purpose of increasing the intelligibility of the claims and, accordingly, have no limiting effect on the interpretation of each element identified by way of example by such reference marks.

1. An integrated system for sanitization and emergency lighting of rooms, comprising:
an external housing (23), which includes a white light source (13) with an optical system, apt to realize an emergency lighting, and a battery (18) for powering said emergency lighting in the absence of a main power supply,
wherein said integrated system also comprises
a chamber (19), provided with reflecting walls and internal shade baffles (16);
a fan (22), of axial or tangential type, which is configured to create inside said chamber (19) a circulation or flow of forced air (F) taken from outside, upstream of said chamber (19), through inlet vents (17). and reintroduced outside, downstream of the chamber (19), through outlet vents (10) and
an electronic control circuit (14) comprising a microprocessor (28), which controls the operation of the white light source (13), the battery (18) and the fan (22),
said integrated system being characterized in comprising, housed in said chamber (19):
a catalytic reactor (20) and/or a UVC light source (200) comprising a catalyst and a UVA-UVB source, controlled by said electronic control circuit (14);
an air quality sensor (31) being positioned in proximity to said inlet vents (17) and being adapted to measure the average concentration of volatile organic compounds or VOCs in an environment;
an ambient light sensor (11) being suitable for measuring the brightness of the environment in which it is positioned, said ambient light sensor (11) being used as a sensor of movement of people within the room based on detecting changes in environmental brightness,
wherein said microprocessor (28),
by means of said ambient light sensor (11), is adapted to detect the entry of an occupant into the room and to activate said UVC source when said entry occurs
and, by means of said sensor (31), is configured to turn on the photo-catalytic reactor (20) automatically when the concentration of VOCs exceeds a certain value in the environment, which also typically corresponds to the prolonged presence of people in a room that is not well ventilated.

2. An Integrated system as claimed in claim 1, characterized in that said photo-catalytic reactor (20) consists of an array of UVA-UVB LED plates (20A) associated with planar photo-catalytic plates (20B).

3. An integrated system as claimed in claim 1, characterized in that said UVC light source consists of a low-pressure mercury discharge tube or lamp (200) or a strip containing an array of UVC LEDs (250) having emission wavelengths substantially equal to 270-280 nm.

4. An integrated system as claimed in claim 1, characterized in that said electronic control circuit (14) also comprises
a power supply and/or battery charger block (27),
one or more signaling LEDs (15) and
a radio transceiver (26) or a wired communication interface that receives commands from a centralized control system.

5. An integrated system as claimed in claim 4, characterized in that said microprocessor (28) accomplishes the following functions:
turning on and off said photo-catalytic reactor (20) and/or said UVC source and regulating the power thereof;
switching on and off said white light source (13) of emergency lighting;
controlling said power supply and battery charger block (27) and determining the status of said battery (18) according to the activation of said emergency lighting;
piloting said one or more signaling LEDs (15), in order to indicate to the user a series of states, such as power supply present, photo-catalytic reactor (20) on and/or UVC source on, and/or failures or malfunctions, emergency lighting failure;
managing said radio transceiver (26) for communication with external devices, such as smartphones or tablets, for configuration and control.

6. An integrated system as claimed in claim 4, characterized in that said microprocessor (28) manages the speed of said fan (22), so as to control the speed of said airflow (F).

7. An integrated system as claimed in claim 1, characterized in that said microprocessor (28) has an astronomical clock, calibrated at the factory and having its own battery, for activating said photo-catalytic reactor (20) and said UVC light source.

8. An integrated system as claimed in claim 3, characterized in that a temperature sensor (240) is placed on said discharge tube or lamp (200) and said microprocessor (28) is configured to measure the temperature of said discharge tube or lamp (200) and to control the driving power of said discharge tube or lamp (200) so as to maintain said temperature at a predetermined level.

9. An integrated system as claimed in claim 1, characterized in that said microprocessor (28) cyclically activates said photo-catalytic reactor (20) and said UVC light source.

10. An integrated system as claimed in claim 1, characterized in comprising an ozone generator (32), which is activated by said microprocessor (28) upon exceeding a certain overall concentration of volatile organic compounds detected by said air quality sensor (31).

11. An integrated system as claimed in claim 10, characterized in that said ozone generator (32) is activated by a command given by a smartphone equipped with a suitable APP or by means of a radio remote centralized emergency lighting control command, possibly cloud-based or cyclically and/or by means of a daily or weekly timer programmed during configuration of the system.

12. An integrated system as claimed in claim 10, characterized in that said ozone generator (32) is kept off until the presence of people in the room is detected via one or more microwave and/or pyroelectric sensors (24, 25).

13. An integrated system as claimed in claim 10, characterized in that said ozone generator (32) is activated only if the presence of people is not detected for a specified time, and if the presence of people is detected, said ozone generator (32) is deactivated and an audible buzzer (38) is activated to indicate to leave the room or ventilate the room.

14. An integrated system as claimed in claim 10, characterized in that said radio transceiver (26) sends to a relay actuator the command to turn on and off an external fan for air exchange in the room at the end of an ignition phase of said ozone generator (32).

15. An integrated system as claimed in claim 1, characterized in that said fan (22) and other parts made of plastic material are protected with staggered metal grids (21), suitable for limiting the intensity of UV radiation directed on said parts.

16. An integrated system as claimed in claim 1, characterized in that, in the absence of a main power supply, said photo-catalytic reactor (20) is turned off and/or said UVC source is turned off, and said battery (18) is used to power said white light source (13) for a defined time.

17. (canceled)

18. (canceled)
